(19) United States
(12) Patent Application Publication
(10) Pub. No.: US 2025/0367031 A1
(43) Pub. Date: Dec. 4, 2025

(54) OPHTHALMIC SURGICAL SYSTEM AND METHOD FOR OPERATING THE OPHTHALMIC SURGICAL SYSTEM

(71) Applicant: Carl Zeiss Meditec AG, Jena (DE)

(72) Inventors: Christoph Kuebler, Oberkochen (DE); Susanne Kohlhammer, Blaustein (DE)

(21) Appl. No.: 19/301,697

(22) Filed: Aug. 15, 2025

Related U.S. Application Data
(63) Continuation of application No. PCT/EP2024/053424, filed on Feb. 12, 2024.

(30) Foreign Application Priority Data
Feb. 15, 2023 (DE) 10 2023 103 654.8

(51) Int. Cl. A61F 9/007; A61M 1/00

(52) U.S. Cl. CPC A61F 9/00745; A61M 1/73; A61M 1/743; A61M 1/772; A61M 1/774; A61B 2217/005; A61B 2217/007; A61M 2205/0216; A61M 2205/3327; A61M 2205/3337; A61M 2205/3344

(57) ABSTRACT

An ophthalmic surgical system includes: a first position sensor for detecting a first deflection position of the first elastic partition element, a second position sensor for detecting a second deflection position of the second elastic partition element, a first pressure sensor for detecting a first pressure in a first drive fluid line, a second pressure sensor for detecting a second pressure at an outlet of a first pump chamber and/or a third pressure sensor for detecting a third pressure at an inlet of a second pump chamber, a fourth pressure sensor for detecting a fourth pressure in the second drive fluid line, a direct connection line configured to connect the irrigation fluid line to the aspiration fluid line, a processing unit for processing the first pressure, the second pressure and/or the third pressure, the fourth pressure, in each case dependent on the first and the second deflection position.

### CROSS-REFERENCE TO RELATED APPLICATIONS

This application is a continuation application of international patent application PCT/EP2024/053424, filed Feb. 12, 2024, designating the United States and claiming priority from German application 10 2023 103 654.8, filed Feb. 15, 2023, and the entire content of both applications is incorporated herein by reference.

### TECHNICAL FIELD

The disclosure relates to an ophthalmic surgical system and to a method for operating the ophthalmic surgical system.

### BACKGROUND

There are a number of surgical techniques for treating clouding of a crystalline lens, which is referred to in medicine as a cataract. The most widespread technique is phacoemulsification, in which a thin hollow needle is introduced into the crystalline lens and is induced to make ultrasonic vibrations. In its immediate surroundings, the vibrating hollow needle emulsifies the lens in such a way that the resulting lens particles can be aspirated through a line via a pump. An irrigation fluid is delivered during this process, with the aspiration of the lens particles and of the fluid taking place through an aspiration fluid line. When the lens has been completely emulsified and removed, a new artificial lens can be inserted into the empty capsular bag, and so a patient treated in this way can recover good vision.

Fluid pumps like in an ophthalmic surgical system in accordance with DE 10 2016 201 297 B3 can be used to enable both the comminution of the crystalline lens with a desired amount of irrigation fluid at the desired pressure and the aspiration of the desired amount of aspiration fluid at the desired pressure. Multiple fluid pumps are used in the process. Since there are multiple fluid pumps that interact with each other and a fluid pump can never be manufactured completely identically to another fluid pump as a matter of principle, it is possible that the fluid pumps do not convey the fluid to be conveyed with the desired accuracy. Already small deviations in the accuracy of the components used in the fluid pumps can lead to unwanted deviations in relation to the fluid to be conveyed, and so a surgical treatment may be difficult.

DE 10 2021 111 178 A1 relates to a method for operating a fluid pump and an ophthalmic surgical system having a fluid pump.

### SUMMARY

It is an object of the disclosure to provide an ophthalmic surgical system with which supplied irrigation fluid and removed aspiration fluid can be controlled with little effort and with high accuracy. It is a further object of the disclosure to provide a method for controlling such an ophthalmic surgical system, which can be performed with little effort.

The object is achieved via various embodiments of the disclosure.

The ophthalmic surgical system includes:
a first fluid pump that includes a first pump chamber having a first volume and a first drive chamber separated therefrom by a first elastic partition element and having a second volume,
a second fluid pump that includes a second pump chamber having a third volume and a second drive chamber separated therefrom by a second elastic partition element and having a fourth volume,
an irrigation fluid line for guiding irrigation fluid to an inlet of the first pump chamber, through the pump chamber to an outlet of the first pump chamber and from there to a first connector configured to be coupled to a surgical instrument,
an aspiration fluid line for guiding aspiration fluid from a second connector configured to be coupled to the surgical instrument to an inlet of the second pump chamber, through the second pump chamber to an outlet of the second pump chamber,
a first drive fluid line for guiding a first drive fluid to the first drive chamber, whereby the second volume can be increased via a deformation of the first elastic partition element and the first volume can be reduced at the same time,
a second drive fluid line for guiding a second drive fluid to the second drive chamber, whereby the fourth volume can be decreased via a deformation of the second elastic partition element and the third volume can be increased at the same time,
a first position sensor for detecting a first deflection position of the first elastic partition element,
a second position sensor for detecting a second deflection position of the second elastic partition element,
a first pressure sensor for detecting a first pressure in the first drive fluid line,
a second pressure sensor for detecting a second pressure at the outlet of the first pump chamber, and/or a third pressure sensor for detecting a third pressure at the inlet of the second pump chamber,
a fourth pressure sensor for detecting a fourth pressure in the second drive fluid line,
a connection line configured to directly connect the irrigation fluid line to the aspiration fluid line,
a processing unit configured to process the first pressure, the second pressure and/or the third pressure, the fourth pressure, in each case in a manner dependent on the first deflection position and the second deflection position.

The acquisition and processing of three or four pressure values and the use of a connection line which directly connects the irrigation fluid line and the aspiration fluid line to each other allows for an accurate evaluation, with little effort, of the actual pressure values setting in at a first fluid pump located in the irrigation fluid line and at a second fluid pump located in the aspiration fluid line. This eliminates the need for the first fluid pump and the second fluid pump to have almost identical properties, and so the requirements for the production of fluid pumps and the components that are coupled to them can be lower than before. The connection line makes it possible to obtain accurate information about the fluidic situation in the ophthalmic surgical system without the influence of a surgical instrument and a patient's eye.

According to various embodiments, the system includes a control unit that acquires signals from the processing unit and is coupled to a first actuator in the first drive fluid line for controlling the first drive fluid and to a second actuator in the second drive fluid line for controlling the second drive fluid. The first actuator allows an accurate supply of the first drive fluid into the first drive chamber. This allows the supply of irrigation fluid from the first pump chamber to be controlled accurately. This also applies analogously to the second actuator and the second drive fluid line and second drive chamber and second pump chamber.

According to various embodiments, the processing unit is configured to process a difference between the first pressure and second pressure as a function of the first deflection position. With knowledge of such a deflection-position-dependent pressure difference, it is possible to precisely set the desired pressure in the irrigation fluid line.

According to various embodiments, the processing unit is configured to process a difference between the fourth pressure and third pressure as a function of the second deflection position. With knowledge of such a deflection-position-dependent pressure difference, it is possible to precisely set the desired pressure in the aspiration fluid line.

It an furthermore be preferred that the processing unit is configured to process a difference between the second pressure and third pressure as a function of a hydraulic resistance of at least a portion of the irrigation fluid line, the connection line and at least a portion of the aspiration fluid line. If the absolute value of this pressure difference is divided by the absolute value for the hydraulic resistance in the aforementioned lines, this results in an absolute value for the flow through the irrigation fluid line, connection line and aspiration fluid line. The knowledge of the flow in the irrigation fluid line and in the aspiration fluid line at the same time is advantageous, for example in order during a surgical treatment to determine and supplement the amount of fluid required in the event of a leakage on the eye.

According to various embodiments, the processing unit is configured to process the flow and a time derivative of the first deflection position. The time derivative of the first deflection position corresponds to a speed in the movement of the first elastic partition element. Should the flow and the associated speed of the first elastic partition element be processed, this enables accurate control of the amount to be supplied by the first fluid pump and the pressure of the irrigation fluid to be applied during a surgical treatment in which a sudden rapid change in flow or pressure occurs in the irrigation fluid line.

The processing unit may also be configured to process the flow and a time derivative of the second deflection position. Should the flow and the associated speed of the second elastic partition element be processed, this enables accurate control of the amount to be removed by the second fluid pump and the pressure of the aspiration fluid to be applied during a surgical treatment in which a sudden rapid change in flow or pressure occurs in the aspiration fluid line.

According to the disclosure, a method for controlling the ophthalmic surgical system described above includes:
closing an outlet valve of the first pump chamber and an inlet valve of the second pump chamber,
filling the first pump chamber with irrigation fluid,
emptying the first drive chamber of the first drive fluid,
emptying the second pump chamber of the aspiration fluid,
filling the second drive chamber with the second drive fluid,
closing an inlet valve of the first pump chamber and an outlet valve of the second pump chamber,
supplying the first drive fluid to the first drive chamber,
connecting the irrigation fluid line to the aspiration fluid line via the connection line,
opening the outlet valve of the first pump chamber and the inlet valve of the second pump chamber,
removing the second drive fluid from the second drive chamber,
emptying the first pump chamber of irrigation fluid and filling the second pump chamber with this irrigation fluid,
acquiring measured values for the first deflection position of the first position sensor, the second deflection position of the second position sensor, the first pressure, the second pressure and/or the third pressure, the fourth pressure,
supplying the measured values to the processing unit.

According to various embodiments, the values ascertained by the processing unit are transmitted to a control unit which is coupled to a first actuator in the first drive fluid line for controlling the first drive fluid and to a second actuator in the second drive fluid line for controlling the second drive fluid.

### BRIEF DESCRIPTION OF DRAWINGS

The invention will now be described with reference to the drawings wherein:

FIG. 1 shows a schematic illustration of an embodiment of the ophthalmic surgical system with connectable components;

FIG. 2 shows a first schematic diagram that shows a signal profile of a first differential pressure as a function of measured values from a first position sensor;

FIG. 3 shows a second schematic diagram that shows a signal profile of a second differential pressure as a function of measured values from a second position sensor;

FIG. 4 shows a third schematic diagram that shows a signal profile of a third differential pressure as a function of measured values from the first position sensor; and,

FIG. 5 shows a fourth schematic diagram that shows a signal profile of the third differential pressure as a function of measured values from the second position sensor.

### DETAILED DESCRIPTION

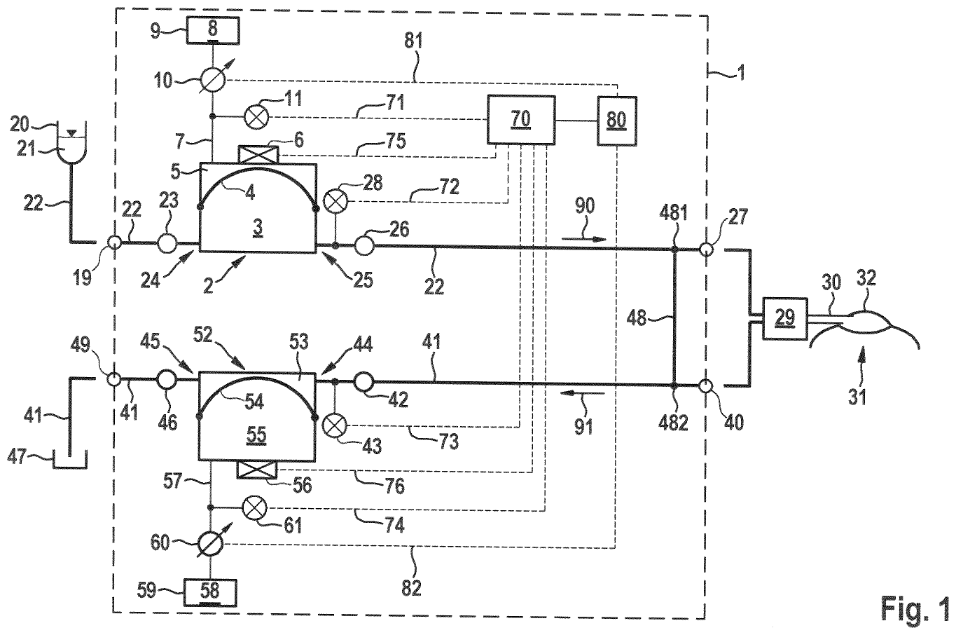

FIG. 1 shows a schematic illustration of an embodiment of an ophthalmic surgical system 1. The system 1 includes a first fluid pump 2 that includes a first pump chamber 3 with a first volume and a first drive chamber 5 with a second volume. The first pump chamber 3 and the first drive chamber 5 are separated from each other by a first elastic partition element 4 such that no exchange of fluid from the first pump chamber 3 to the first drive chamber 5, or vice versa, is possible.

In the first fluid pump 2, the first elastic partition element 4 is permanently attached at its edge region. Should the volume of the first pump chamber 3 be equal to the volume of the first drive chamber 5, the first elastic partition element 4 is in the horizontal position. Should the first volume be greater than the second volume, the first elastic partition element 4 is in an extended position in which it may be, for example, substantially convex; cf. FIG. 1. The first elastic partition element 4 may have any desired geometry and may also adopt different positions in the event of a volume difference between the first pump chamber 3 and the first drive chamber 5. The illustration in FIG. 1 is drawn only schematically and not to scale.

In its central region, the first elastic partition element 4 may include a first element that is suitable for contactless detection via a first position sensor 6. The first position sensor 6 may be an inductive or capacitive position sensor. The first position sensor 6 may be arranged at the edge of the fluid pump 2.

The first drive chamber 5 is connected to a first drive fluid line 7. A first drive fluid 8 can be supplied from a first drive fluid container 9 into the first drive chamber 5, in a manner dependent on a first actuator 10. This process is reversible, and so drive fluid from the first drive chamber 5 can be returned along the drive fluid line 7 to the first drive fluid container 9. The fluid pressure present in the drive fluid line 7 can be detected as the first pressure p1 via a first pressure sensor 11, which is preferably coupled to a connector on the drive fluid line 7.

The first pump chamber 3 can be fed with irrigation fluid 21 at its inlet 24. The irrigation fluid 21 is contained in an irrigation fluid container 20, which can be coupled to the system 1 via a third connector 19 via an irrigation fluid line 22. In that case, the irrigation fluid line 22 is connected to the inlet 24 of the first fluid pump 2. Supply of the irrigation fluid 21 to the first fluid pump 2 can be enabled or blocked via a first inlet valve 23, wherein the first inlet valve 23 belongs to the first pump chamber 3 but need not be directly connected to the first pump chamber 3. Filling the first pump chamber 3 with irrigation fluid 21 requires a first outlet valve 26 arranged downstream of a first outlet 25 to be closed, wherein the first outlet valve 26 belongs to the first pump chamber 3 but need not be connected to the first pump chamber 3.

When the first inlet valve 23 is closed and the first outlet valve 26 is open, the irrigation fluid 21 can be pressed out of the first pump chamber 8 by inflow of the first drive fluid 3 into the first drive chamber 5 and can flow to the first outlet 25 and out into the irrigation fluid line 22 to a first connector 27. Immediately downstream of the first outlet 25 of the first fluid pump 2, the ophthalmic surgical system 1 includes a connector on the irrigation fluid line 22 for a second pressure sensor 28, wherein a second pressure p2 present in the irrigation fluid line 22 can be detected using the second pressure sensor 28. The first connector 27 is configured to be coupled to a line of a surgical instrument 29 such that, in the coupled state, the irrigation fluid can flow to the surgical instrument 29 and can be used for a surgical treatment.

The surgical instrument 29 may include a needle 30 from which irrigation fluid may flow out. The irrigation fluid 21 may be used in a phacoemulsification of a lens 32 of an eye 31.

Should lens particles be removed from the eye during phacoemulsification, they can be aspirated through the needle 30 along an aspiration fluid line 41. To this end, the aspiration fluid line 41 is coupled to the ophthalmic surgical system 1 by way of a second connector 40 such that the aspiration fluid can reach a second inlet 44 of a second pump chamber 53 of a second fluid pump 52 after passing an open second inlet valve 42 arranged in the aspiration fluid line 41. The second inlet valve 42 is an inlet valve that belongs to the second pump chamber 53 but need not be directly connected to the second pump chamber 53. A third pressure p3 in the aspiration fluid line 41 immediately upstream of the second inlet 44 of the second pump chamber 53 can be detected via a third pressure sensor 43, which immediately upstream of the second inlet 44 is coupled to a connector on the aspiration fluid line 41.

The second fluid pump 52 is constructed in a manner analogous to the first fluid pump 2. The second fluid pump 52 includes a second pump chamber 53 and a second drive chamber 55 arranged adjacent thereto, which chambers are separated from each other by a second elastic partition element 54. The second elastic partition element 54 is securely connected at its edge region to the second fluid chamber 52. The position of the second elastic partition element 54 can be detected via a second position sensor 56, which for example is arranged at the edge of the second pump chamber 52 or brought into contact with the latter. The second pump chamber 53 has a third volume, and the second drive chamber 55 has a fourth volume.

The second drive chamber 55 can be emptied or filled with a second drive fluid 58 from a second drive fluid container 59 along a second drive fluid line 57. The drive fluid flow is controlled via a second actuator 60. A fourth pressure p4 can be detected via a fourth pressure sensor 61, which is coupled to the drive fluid line 57 via a connector.

Should drive fluid be conveyed from the second drive chamber 55 in the direction of the drive fluid container 59, aspiration fluid can flow into the second pump chamber 53 on account of the pressure equalization. Should a second outlet valve 46 arranged downstream of a second outlet 45 of the second fluid pump 52 and in the aspiration fluid line 41 be closed, the third volume of the second pump chamber 53 increases when aspiration fluid flows in, with the fourth volume of the second drive chamber reducing at the same time. The second outlet valve belongs to the second pump chamber 53 but need not be directly connected to the second pump chamber 53. The second elastic partition element 54 deforms in the process. Should the second inlet valve 42 be closed and the second outlet valve 46 be open, the aspiration fluid located in the second pump chamber 53 can flow into the aspiration fluid line 41 and then into a drive fluid collection container 47 by filling the second drive chamber 55 with drive fluid 58.

The ophthalmic surgical system 1 furthermore includes a connection line 48 configured to directly connect the irrigation fluid line 22 to the aspiration fluid line 41. A first end 481 of the connection line 48 may be connected to the first connector 27 and a second end 482 of the connection line 48 may be connected to the second connector 40, with the system 1 in that case being configured such that no fluid can flow to a surgical instrument 29. Alternatively, the first end 481 of the connection line 48 may be arranged between the connector on the irrigation fluid line 22 for the first pressure sensor 28 and the first connector 27, and the second end 482 of the connection line 48 may be arranged between the second connector 40 and the connector on the aspiration fluid line 41 for the third pressure sensor 43, with the system in that case likewise being configured such that no fluid can flow to the surgical instrument 29. This embodiment is illustrated in FIG. 1. It is also possible that the first end 481 of the connection line 48 is coupled to the irrigation fluid line 22 upstream of the first inlet 24, as seen in the flow direction 90, and the other end 482 of the connection line 48 with the aspiration fluid line 41 is arranged downstream of the second outlet 45, as seen in the flow direction 91. In this case, it must be ensured that no fluid flows downstream of the first outlet 25 in the flow direction 90 and that no fluid flows toward the second inlet 44 in the flow direction 91. This can be achieved by the first outlet valve 26 and the second inlet valve 42 being closed.

The connection line 48 is thus configured to connect the irrigation fluid line 22 to the aspiration fluid line 41, with the system 1 being configured to prevent a fluidic connection to the surgical instrument 29 at the same time.

The connection line 48 acts as a direct connection line or “short-circuit line”. The system 1 is thus configured such that between the irrigation fluid line 22 and the aspiration fluid line 41 there is no other direct connection line in which fluid could flow along a “detour” or a line extending parallel thereto.

During ophthalmic surgical treatment, irrigation fluid can flow into a connected surgical instrument 29 and out of the instrument 29 again as aspiration fluid. In this case, the system 1 is configured such that no fluid can flow through the connection line 48. The system 1 is configured such that a flow of fluid through the connection line 48 is only possible before or after a surgical treatment but not during a surgical treatment.

An irrigation fluid line 22 is understood to be a fluid line through which fluid from the irrigation fluid container 20 can flow to the third connector 19, from there to the first inlet 24 of the first fluid pump 2, then through the first pump chamber 3, and out of the first outlet 25 through the first outlet valve 26 to the first connector 27. When a surgical instrument 29 is connected, the irrigation fluid line 22 also includes the line up to the surgical instrument 29 and up to an outlet of the surgical instrument 29.

An aspiration fluid line is understood to be a fluid line through which fluid from a surgical instrument 29 possibly connected to the second connector 40 can flow to the second connector 40, from there to the second inlet valve 42 and to the second inlet of the second pump chamber 53, through the second pump chamber 53 to the second outlet 45 up to the second outlet valve 46 and from there to the aspiration fluid collection container 47.

The surgical system 1 additionally includes a processing unit 70. The processing unit 70 is configured to receive and process signals from the first pressure sensor 11 via a first signal line 71 and from the second pressure sensor 28 via a second signal line 72. In addition, the processing unit 70 is configured to receive and process signals from the third pressure sensor 43 via a third signal line 73 and from the fourth pressure sensor 61 via a fourth signal line 74. Moreover, the processing unit 70 is configured to receive and process signals from the first position sensor 6 via a fifth signal line 75 and from the second position sensor 56 via a sixth signal line 76. The processing unit 70 is connected to a control unit 80 of the ophthalmic surgical system 1, and so a result of processing the signals from the pressure sensors and position sensors can be used to control the first actuator 10 via a seventh signal line 81 and the second actuator 61 via an eighth signal line 82.

FIG. 1 shows the ophthalmic surgical system 1, in which the irrigation fluid container 20 with irrigation fluid 21 and a portion of the irrigation fluid line 22 are not coupled to the third connector 19. Furthermore, the aspiration fluid collection container 47 with a portion of the aspiration fluid line 41 is not coupled to the fourth connector 49. The surgical handpiece 29 is not coupled to the first connector 27 and the second connector 40. However, with its first end 481 the connection line 48 is coupled to the irrigation fluid line 22, and the second end 482 of the connection line 48 is coupled to the aspiration fluid line 49.

If a surgical treatment such as a phacoemulsification should be performed, the irrigation fluid container 20 with irrigation fluid 21 and the portion of the irrigation fluid line 22 are coupled to the third connector 19. Likewise, the aspiration fluid collection container 47 with the portion of the aspiration fluid line 41 is coupled to the fourth connector 49. Moreover, the surgical handpiece 29 is coupled to the first connector 27 and the second connector 40. In that case, however, the first end 481 of connection line 48 is not coupled to the irrigation fluid line 22 and/or its second end 482 is not coupled to the aspiration fluid line 41.

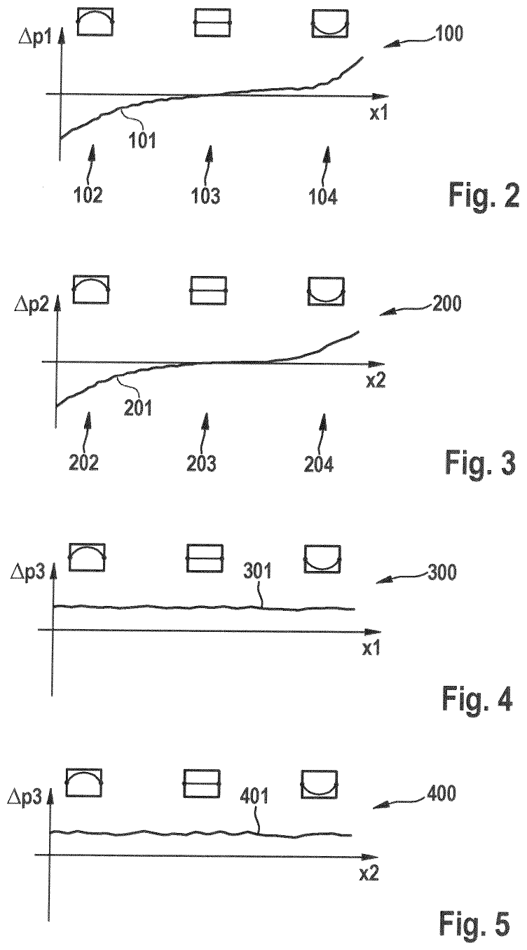

The processing of the signals from the pressure sensors and position sensors is explained below with the aid of FIGS. 2 to 5. The graphs shown in these figures have been ascertained in the state in which the connection line 48 connects the irrigation fluid line 22 and the aspiration fluid line 41 to each other, and no fluid can flow to a surgical instrument 29. The graphs represent calibration curves, which can preferably be recorded before surgical treatment.

When the connection line 48 directly connects the irrigation fluid line 22 to the aspiration fluid line 41 and no surgical handle is used, no particles of an emulsified crystalline lens 32 and no other fluid, which comes from an eye 31 of a patient, flow in the aspiration fluid line. Instead, the irrigation fluid 21 from the irrigation fluid line 22 flows in the aspiration fluid line 41.

FIG. 2 shows a first diagram 100 with a first graph 101 under the following conditions:
the first inlet valve 23 is closed;
the first pump chamber 3 is filled with irrigation fluid 21;
the first drive chamber 5 is not filled with any drive fluid 8;
the first outlet valve 26 is open;
the connection line 48 connects the irrigation fluid line 22 to the aspiration fluid line 41;
no fluid can flow to a surgical instrument;
the second inlet valve 42 is open;
the second pump chamber 53 contains no fluid;
the second drive chamber 55 is completely filled with drive fluid 58;
the second outlet valve 46 is closed;
the first drive fluid 8 can flow from the first drive fluid container 9 into the first drive fluid line 7;
from the second drive chamber 55, drive fluid 58 can flow to the second drive fluid container 59.

The first elastic partition element 4 and the second elastic partition element 54 are in corresponding positions, for example both partition elements 4 and 54 are initially convexly shaped, as shown in FIG. 1. In FIGS. 2 to 5, therefore, the respective partition element is plotted symbolically in convex form in the left part of the diagrams. In the respective middle part, they are plotted in a relaxed horizontal position, and in the respective right part they are plotted in a concave position.

In the diagram 100, a difference Δp1 between the first pressure p1 and the second pressure p2 is plotted on the ordinate. The displacement x1 detected by the first position sensor 6 is plotted on the abscissa. The left region 102 in the graph 101 shows difference values that are smaller than zero. This may be explained as follows. The first elastic partition element 4 and the second elastic partition element 54 are in a very strongly deformed convex position and have a high restoring force in the direction of a relaxed position. Only a relatively small amount of the first drive fluid 8 is necessary to press the irrigation fluid out of the first pump chamber 3 in the direction of the first outlet valve 26. The first pressure p1 is therefore slightly lower than the second pressure p2, and so the difference Δp1=p1−p2 is negative.

In a middle region 103 of graph 101, the elastic partition elements 4 and 54 are in an approximately horizontal position and are only slightly deformed, or not deformed at all, and thus relatively relaxed. In this case, the first pressure p1 for the first drive fluid 8 is nearly the same as or identical to the second pressure p2 of the irrigation fluid. The difference Δp1 is therefore virtually zero or equal to zero in the middle region 103 of graph 101.

The values are positive in a right region 104 in the first graph 101. Relatively large amounts of pressure must be exerted by the first drive fluid 8 on the first elastic partition element 4 in order to bring this partition element 4 into a relatively strongly concavely deformed position. In this situation, irrigation fluid 21 flows out of the first outlet 25 at a lower pressure. The difference Δp1=p1−p2 is therefore positive.

Multiplying the pressure difference Δp1 by the projected cross-sectional area of the first pump chamber 3 results in a compressive force. Hence, a force-displacement characteristic curve for the first elastic partition element 4 can be determined via the first graph 101. It makes sense to record the force-displacement characteristic curve for the entire travel of the elastic partition element 4. This corresponds to the situation where the pump chamber 3 is initially completely filled with irrigation fluid 21 and completely empty at the end of the movement of the partition element 4.

FIG. 3 shows a second diagram 200 with a second graph 201 under the same conditions as those indicated above in relation to FIG. 2. The ordinate plots a difference Δp2 between the fourth pressure p4 and the third pressure p3. The displacement x2 detected by the second position sensor 56 is plotted on the abscissa. The left region 202 in the second graph 201 shows difference values that are smaller than zero. The second elastic partition element 54 is strongly convexly deformed and has a high restoring force in the direction of a relaxed position. Therefore, a relatively low pressure needs to be applied in the second drive fluid line 57. The fourth pressure p4 is therefore lower in terms of absolute value than the third pressure p3 of the fluid flowing into the second pump chamber 53 at the second inlet 44. Hence, Δp2=p4−p3 is negative; see left region 202 in the graph 201.

In the middle region 203 in the second graph 201, the second elastic partition element 54 is in a slightly deformed or not at all deformed position. Hence, the fourth pressure p4 is virtually equal or equal to the third pressure p3, and the difference Δp2 is virtually zero or equal to zero.

The values are positive in the right region 204 in the second graph 201. To bring the second elastic partition element 54 into a concave shape, a relatively strong negative pressure must be applied in the second drive fluid line 57. Since the third pressure p3 is also a negative pressure, Δp2=p4−p3 is therefore positive.

Multiplying the second pressure difference by the projected cross-sectional area of the second pump chamber 53 results in a compressive force. Hence, it is possible to ascertain a force-displacement characteristic curve for the second elastic pressure element 54.

The first elastic partition element 4 and the second elastic partition element 54 are two different components. They can be manufactured with great precision but are not identical. This also applies analogously to the first drive fluid line 7 and the second drive fluid line 57. Likewise, the first pressure sensor 11 and the fourth pressure sensor 43 are two different components that do not provide identical measured values. It should therefore be expected that in an accurate representation the first graph 101 is not exactly the same as the second graph 201.

FIG. 4 shows a third diagram 300 with a third graph 301. In the diagram 300, a difference Δp3 between the second pressure p2 and the third pressure p3 is plotted on the ordinate, that is, Δp3=p2−p3. The displacement x1 detected by the first position sensor 6 is plotted on the abscissa.

The second pressure p2 is the pressure in the irrigation fluid line 22 immediately downstream of the first outlet 25 of the first pump chamber 3, and the third pressure p3 is the pressure in the aspiration fluid line 41 immediately upstream of the second inlet 44 of the second pump chamber 53. The length of line between the connector of the second pressure sensor 28 to the first end 481 of the connection line 48, the connection line 48 to the second end 482 of the connection line 48 and from there to the connector of the third pressure sensor 43 has a hydraulic resistance greater than zero. This causes the third pressure p3 to be slightly lower than the second pressure p2. This applies to the entire travel of the first elastic partition element 4, and so the third graph 302 shows a positive differential pressure Δp3 along the entire path x1. Should this differential pressure Δp3 be divided by the hydraulic resistance R, this corresponds to the fluid flow through the specified section.

FIG. 5 shows a fourth diagram 400 with a fourth graph 401. In this diagram 400, the difference Δp3 between the second pressure p2 and the third pressure p3 is plotted on the ordinate, that is, Δp3=p2−p3. The displacement x2 detected by the second position sensor 56 is plotted on the abscissa. The only difference to FIG. 4 thus consists in the fact that the pressure difference is shown as a function of not the first position sensor 6 but the second position sensor 56. Should this pressure difference be divided by the hydraulic resistance of the length from the connector of the second pressure sensor 28 to the third position sensor 43, this corresponds to the fluid flow through this length.

It is possible that the hydraulic resistance R of a length running from the connector of the second pressure sensor 28 to the first end 481 of the connection line 48, the connection line 48 to the second end 482 of the connection line 48 and from there to the connector of the third pressure sensor 43 is known and that the fluid flow Q along this length is known too. In that case, it is possible to omit either the second pressure sensor 28 or the third pressure sensor 43. If the second pressure sensor 28 is present and a second pressure p2 is available but no third pressure sensor 43 or third pressure p3 is known, the third pressure is calculated as p3=p2−Q*R. In that case, the third pressure p3 is available not as a measured value but as a calculation value, which can be ascertained by the processing unit 70. By contrast, if the third pressure sensor 43 and the corresponding third pressure p3 are known but no second pressure sensor 28 is present or no second pressure p2 is available, the second pressure is calculated as p2=p3+Q*R, wherein this second pressure can be ascertained by the processing unit 70.

Using the system 1, it is thus possible to ascertain the spring characteristic curve of the first elastic partition element 4 and the associated flow through the irrigation fluid line 22 for a calibration from the one-time movement of the first elastic partition element 4 from a filled first pump chamber 3 to an emptied first pump chamber 3. This also applies analogously to the second elastic partition element 54. Since this calibration for the first elastic partition element 4 and the second elastic partition element 54 may be performed simultaneously, four characteristic curves can be determined after only one one-time movement sequence from the filled first pump chamber 3 to the emptied first pump chamber 3 or from the emptied second pump chamber 53 to the filled second pump chamber 53. This means a significant time saving in comparison with conventional calibration procedures, where each pump chamber must be calibrated individually and sequentially in time.

It is understood that the foregoing description is that of the preferred embodiments of the invention and that various changes and modifications may be made thereto without departing from the spirit and scope of the invention as defined in the appended claims.

### REFERENCE SIGNS

1 Ophthalmic surgical system
2 First fluid pump
3 First pump chamber
4 First elastic partition element
5 First drive chamber
6 First position sensor
7 First drive fluid line
8 First drive fluid
9 First drive fluid container
10 First actuator
11 First pressure sensor
19 Third connector
20 Irrigation fluid container
21 Irrigation fluid
22 Irrigation fluid line
23 First inlet valve of the first pump chamber
24 First inlet of the first pump chamber
25 First outlet of the first pump chamber
26 First outlet valve of the first pump chamber
27 First connector
28 Second pressure sensor
29 Surgical instrument
30 Hollow needle
31 Eye
32 Lens
40 Second connector
41 Aspiration fluid line
42 Second inlet valve of the second pump chamber
43 Third pressure sensor
44 Second inlet of the second pump chamber
45 Second outlet of the second pump chamber
46 Second outlet valve of the second pump chamber
47 Aspiration fluid collection container
48 Connection line
481 First end of the connection line
482 Second end of the connection line
49 Fourth connector
52 Second fluid pump
53 Second pump chamber
54 Second elastic partition element
55 Second drive chamber
56 Second position sensor
57 Second drive fluid line
58 Second drive fluid
59 Second drive fluid container
60 Second actuator
61 Fourth pressure sensor
70 Processing unit
71 First signal line
72 Second signal line
73 Third signal line
74 Fourth signal line
75 Fifth signal line
76 Sixth signal line
80 Control unit
81 Seventh signal line
82 Eighth signal line
90 Flow direction
91 Flow direction
100 First diagram
101 First graph
102 Left region in the first graph
103 Middle region in the first graph
104 Right region in the first graph
200 Second diagram
201 Second graph
202 Left region in the second graph
203 Middle region in the second graph
204 Right region in the second graph
300 Third diagram
301 Third graph
400 Fourth diagram
401 Fourth graph
p1 First pressure
p2 Second pressure
p3 Third pressure
p4 Fourth pressure
Δp1 First differential pressure
Δp2 Second differential pressure
Δp3 Third differential pressure
Q Fluid flow rate
R Hydraulic resistance

1. An ophthalmic surgical system comprising:
a first fluid pump including a first pump chamber having a first volume and a first drive chamber separated therefrom by a first elastic partition element and having a second volume;
a second fluid pump including a second pump chamber having a third volume and a second drive chamber separated therefrom by a second elastic partition element and having a fourth volume;
an irrigation fluid line for guiding irrigation fluid to an inlet of said first pump chamber, through said first pump chamber to an outlet of said first pump chamber and from there to a first connector configured to be coupled to a surgical instrument;
an aspiration fluid line for guiding aspiration fluid from a second connector configured to be coupled to the surgical instrument to an inlet of said second pump chamber, through said second pump chamber to an outlet of said second pump chamber;
a first drive fluid line for guiding a first drive fluid to said first drive chamber, whereby said second volume is increaseable via a deformation of said first elastic partition element and said first volume is contemporaneously reduceable;
a second drive fluid line for guiding a second drive fluid to said second drive chamber, whereby the fourth volume is decreaseable via a deformation of said second elastic partition element and the third volume is contemporaneously increaseable;
a first position sensor for detecting a first deflection position of said first elastic partition element;
a second position sensor for detecting a second deflection position of said second elastic partition element;
a first pressure sensor for detecting a first pressure in said first drive fluid line;
at least one of a second pressure sensor for detecting a second pressure at said outlet of said first pump chamber and a third pressure sensor for detecting a third pressure at said inlet of said second pump chamber;
a fourth pressure sensor for detecting a fourth pressure in said second drive fluid line;
a connection line configured to directly connect said irrigation fluid line to said aspiration fluid line; and,
a processing unit configured to process the first pressure, the second pressure and/or the third pressure, the fourth pressure, in each case in a manner dependent on said first deflection position and said second deflection position.

2. The ophthalmic surgical system of claim 1 further comprising a control unit configured to acquire signals from said processing unit; and, said control unit being coupled to a first actuator in said first drive fluid line for controlling the first drive fluid and to a second actuator in said second drive fluid line for controlling the second drive fluid.

3. The ophthalmic surgical system of claim 1, wherein said processing unit is configured to process a difference between the first pressure and second pressure as a function of the first deflection position.

4. The ophthalmic surgical system of claim 1, wherein said processing unit is configured to process a difference between the fourth pressure and third pressure as a function of said second deflection position.

5. The ophthalmic surgical system of claim 1, wherein said processing unit is configured to process a difference between the second pressure and the third pressure as a function of a hydraulic resistance of at least a portion of said irrigation fluid line, said connection line and at least a portion of said aspiration fluid line.

6. The ophthalmic surgical system of claim 5, wherein said processing unit is configured to process a flow and a time derivative of the first deflection position together.

7. The ophthalmic surgical system of claim 5, wherein said processing unit is configured to process a flow and a time derivative of the second deflection position together.

8. A method for controlling the ophthalmic surgical system, the ophthalmic surgical system including a first fluid pump including a first pump chamber having a first volume and a first drive chamber separated therefrom by a first elastic partition element and having a second volume, the system further including a second fluid pump including a second pump chamber having a third volume and a second drive chamber separated therefrom by a second elastic partition element and having a fourth volume, the system further including an irrigation fluid line, an aspiration fluid line, a first drive fluid line, a second drive fluid line, a first position sensor, a second position sensor, a first pressure sensor, at least one of a second pressure sensor and a third pressure sensor, a fourth pressure sensor, a connecting line, and a processing unit, the irrigation fluid line being for guiding irrigation fluid to an inlet of the first pump chamber, through said first pump chamber to an outlet of the first pump chamber and from there to a first connector configured to be coupled to a surgical instrument, the aspiration fluid line being for guiding aspiration fluid from a second connector configured to be coupled to the surgical instrument to an inlet of the second pump chamber, through the second pump chamber to an outlet of the second pump chamber; the first drive fluid line being for guiding a first drive fluid to the first drive chamber, whereby the second volume is increaseable via a deformation of the first elastic partition element and the first volume is contemporaneously reduceable; the second drive fluid line being for guiding a second drive fluid to the second drive chamber, whereby the fourth volume is decreaseable via a deformation of the second elastic partition element and the third volume is contemporaneously increaseable, the first position sensor being for detecting a first deflection position of the first elastic partition element, the second position sensor being for detecting a second deflection position of the second elastic partition element, the first pressure sensor being for detecting a first pressure in the first drive fluid line; the at least one of a second pressure sensor being for detecting a second pressure at the outlet of the first pump chamber and the third pressure sensor being for detecting a third pressure at the inlet of the second pump chamber, the fourth pressure sensor being for detecting a fourth pressure in the second drive fluid line, the connection line being configured to directly connect the irrigation fluid line to the aspiration fluid line, and, the processing unit being configured to process the first pressure, the second pressure and/or the third pressure, the fourth pressure, in each case in a manner dependent on the first deflection position and the second deflection position, the method comprising:
closing an outlet valve of the first pump chamber and an inlet valve of the second pump chamber;
filling the first pump chamber with the irrigation fluid;
emptying the first drive chamber of the first drive fluid;
emptying the second pump chamber of the aspiration fluid;
filling the second drive chamber with the second drive fluid;
closing an inlet valve of the first pump chamber and an outlet valve of the second pump chamber;
supplying the first drive fluid to the first drive chamber;
connecting the irrigation fluid line to the aspiration fluid line via the connection line;
opening the outlet valve of the first pump chamber and the inlet valve of the second pump chamber;
removing the second drive fluid from the second drive chamber;
emptying the first pump chamber of irrigation fluid and filling the second pump chamber with this irrigation fluid;
acquiring measured values for the first deflection position of the first position sensor, the second deflection position of the second position sensor, the first pressure, the second pressure and/or the third pressure, the fourth pressure; and,
supplying the measured values to the processing unit.
